(19) United States
(12) Patent Application Publication
(10) Pub. No.: US 2011/0280977 A1
(43) Pub. Date: Nov. 17, 2011

(54) COMPOSITION FOR RELIEVING AND PREVENTING HANGOVER AND BEVERAGE COMPRISING COMPOSITION

(76) Inventors: Hyun-Jun PARK, Busan (KR); Seok-Jun Park, Seoul (KR)

(21) Appl. No.: 13/084,994

(22) Filed: Apr. 12, 2011

(30) Foreign Application Priority Data
Apr. 16, 2010 (KR) 10-2010-035426

(51) Int. Cl. A61K 36/738; A61P 43/00

(52) U.S. Cl. 424/765

(57) ABSTRACT

Disclosed is a composition for relieving and preventing hangover and, more specifically, a composition for relieving and preventing hangover, which includes Hovenia dulcis Thunb fruit extract, Rosa Roxburghii extract, Engelgardtia chrysolepis HANCE extract, rotus (Nelumbo nucifera Gaertner fruits) extract and nicotinic acid amide as active ingredients, and accelerates in vivo absorption/decomposition of alcohol after drinking, thereby helping to relieve and prevent hangover, as well as a beverage for relieving and preventing hangover including the same.

### BACKGROUND OF THE INVENTION

1. Field of the Invention

The present invention relates to a composition for relieving and preventing hangover and, more specifically, a composition for relieving and preventing hangover, which includes Hovenia dulcis Thunb fruit extract concentrate (or extract), Rosa Roxburghii extract, Engelgardtia chrysolepis HANCE extract, Nelumbo nucifera Gaertner extract and nicotinic acid amide as active ingredients, and is absorbed/decomposed in vivo and decreases concentration of blood alcohol and/or acetaldehyde causing alcohol hangover, thereby helping to relieve and prevent hangover, as well as a beverage for relieving and preventing hangover including the same.

2. Description of the Related Art

Alcohol hangover refers to unpleasant physical and/or mental conditions experienced after drinking too much alcohol and objectively includes the following symptoms; decreased consciousness, nausea, vomiting, drowsiness, headache, decreased ability to exercise, hematological variations, hormone changes, or the like.

Modern humans are exposed to a variety of stress during daily life and the amount of stress that people experience continues to increase in the modern society despite of technological advancement.

Although many people attempt to overcome such stress in different ways such as exercise, sleeping, smoking, taking trips, or the like, the most common method in the modern society to relieve stress may be the drinking (alcohol).

The major reason for this is that, for modern people, drinking is not only a simple solution to overcoming stress but is also recognized as a culture obligation in a broad range of circumstances including, for example, improvement in business relationship through entertainment (or with a special service), bonding with co-workers and/or companions, family reunions, or the like.

In particular, as the society becomes more complex and organized, frequent opportunities to drink are encountered, which in turn encourages heavy drinking and/or binge drinking. Since Koreans are obsessed with drinking and alcohol abuse due to Korean traditional drinking culture, Koreans often have hangovers and difficulty in recovering a normal condition or working the next day. Moreover, bad drinking habits continued for a long term can lead to health problems over time.

Ethanol absorbed into the body is mostly metabolized in the liver to form acetaldehyde, in turn being oxidized and discharged in a form of acetate. Oxidation from ethanol to acetaldehyde involves an enzyme, that is, alcohol dehydrogenase (ADH), while decomposition/oxidation of acetaldehyde generated from alcohol oxidation employs another enzyme named aldehyde dehydrogenase (ALDH).

According to a normal alcohol metabolism, alcohol after drinking is absorbed in the stomach or the small intestine and passes through blood vessels into the liver. In liver cells, alcohol is oxidized into acetaldehyde by ADH and acetaldehyde is decomposed into acetate by ALDH, in turn being decomposed into CO₂ gas and water then discharged outside.

Although the exact cause of hangovers is not known, a number of suppositions such as toxicity of alcohol and alcohol metabolites (acetaldehyde, formaldehyde, acetone, etc.), nutrient deficiency caused by nutrient absorption disorders, and so forth have been proposed. In recent years, formaldehyde in alcohol and metabolism of methanol contained in a small amount in alcohol have been indicated as causes of alcohol hangover (Jone A W. Pharmaco Toxicology. 60(3) 217-220). However, some documents have reported that hangover is caused by alcohol metabolites such as acetaldehyde, acetone, or the like (Tomita Y, Haseba T, Kurosu M, Watanabe T (1990), Arukoru Kenkyut Yakubutsu Ison. 25(2). 116-128; Tsukamoto S, Muto T. Nagoya T, Shimamura M, Saito T, Tainaka H. (1989), Alcohol alcohol. 24(2). 101-108).

Acetaldehyde generally includes Type 2 acetaldehyde to begin to be oxidized even when a concentration of in vivo acetaldehyde is low, and Type 1 acetaldehyde to begin to be oxidized only when a concentration of in vivo acetaldehyde is high. Type 2 acetaldehyde commonly generated in Asians is slowly oxidized, thus more frequently causing hangovers, as compared to Type 1 acetaldehyde generated in most westerners.

Because of social grounds of Korean traditional drinking culture, consumers still need improved products for relieving hangovers and extensive studies and investigations into methods for relieving hangovers have been conducted in various applications. In fact, a variety of products are commercially available and/or have recently been introduced on the market.

However, although most products contain herbal materials and/or other compositions known to have bioactive efficiency, contents thereof are substantially too small. Also, since objective verification of efficacy of final products manufactured in the art is still inadequate, reliability of such products assessed by consumers who need effective hangover relief drinks is relatively low.

Accordingly, there is a strong need to develop an improved product having advantages of high reliability, decrease in social loss caused by heavy drinking, contribution to healthier lifestyles of individuals, or the like, by preparing the product from specific raw materials and/or compositions with advantageous effects for hangover relief and subjecting the prepared product to beverage tasting such that actual consumers directly drink the product and experience beneficial effects of the same, in turn assessing and verifying objective hangover relieving efficacy of the product.

Hovenia dulcis Thunb is a Rhamnaceae plant (Koreana Nakai, Japanese Raisin tree, etc.), often referred to as ‘Hutgae tree’ in Korean and provides fresh fruits fixed to stalks thereof. Such fruits are often called ‘Hovenia dulcis,’ and have been used as a medicinal herb since ancient times. Hovenial dulcis has a sweet taste, mild characteristics and various effects such as thirst quenching, removal of stagnated heat, removal of damp-heat, treatment of alcohol poisoning, or the like. The Korean name ‘Hutgae’ is derived from the meaning that “wine comes to nothing.” According to traditional oriental medical texts such as Compendium of Materia Medica (Chinese: B\check{e}nc\check{a}o Gāngmù), which is a pharmaceutical text written by Li Shizhen (1518 to 1593 AD) during the Ming Dynasty of China, Hovenia dulcis is known as the most effective medical herb to treat alcohol poisoning.

In recent years, extensive studies into physiological activity of Hovenia dulcis Thunb have been conducted in various applications and a number of research documents practically demonstrated objective verification of alcohol decomposition and/or liver detoxification effects by Hovenia dulcis Thunb.

Examples of case studies may include: alcohol decomposition by Hovenia dulcis Thunb constituents (Kim. S. M., Kang S. H., et al., Korean Journal of Biotechnology and Bioengineering, Vol. 21, No. 1, 11-15 (2006); Ko B. S., et al., Korean Journal of Food Science and Nutrition 35(7), 828-834 (2006); Ahn S. W., et al., Korean Medicinal Herb 7(4), 263-268 (1999)), Liver detoxification (Kim M. H., et al., Korean Medicinal Herb 8(3), 225-233 (2000); Lim M. K., et al., Korean Journal of Veterinary Science 24(3), 384-391 (2007)), Toxic effects of ethanol upon learning ability (Lim J. P., et al., Korean Medicinal Herb 11(3), 232-235 (2003), or the like.

The present inventors have already suggested objective hangover relieving effects of a composition prepared using extracts of Rosa Roxburghii, Engelgardtia chrysolepis HANCE and Nelumbo nucifera, which was granted Korean Patent No. 10-0620093. However, considering consumer opinion and social needs for development of improved hangover relief drinks and compositions, the present invention has been proposed.

Rosa Roxhurghii is a Rosaceae plant and often called a ‘sweet chestnut rose.’ This plant is distributed throughout The East Asia, China and the Himalayas, and fruits and seeds thereof are edible. Rosa Roxhurghii native to China has been used as a health food supplement and/or beauty food in Guizhou province of China and, in particular, is used to prepare beauty beverages to remove age spots, freckles, wrinkles, etc. in Japan since it contains abundant vitamins or the like and has high SOD activity.

Korean Patent No. 003413 discloses a method for preparation of an anti-stress rose yogurt using rose fruit extract (RFE) and Korean Patent No. 0040338 discloses a method for manufacturing an anti-stress drink effective to inhibit generation of active oxygen (that is, oxygen free radicals) and remove the same using RFE.

Engelgardtia chrysolepis HANCE is a Juglandaceae plant and generally called ‘Taiwan engelhardtia.’ Engelgardtia chrysolepis HANCE is common throughout southern and south-western China and leaves thereof are edible. Since these leaves are slightly sweet thus being edible as a sweet tea and used as a health supplement tea in China since early times. At present, the foregoing plant is often called ‘Kohki tea’ in Japan or the like and is present in a variety of beverages and teas.

Nelumbo nucifera is a Nymphaeaceae plant, generally cultivated in ponds in south central regions and, in view of geographical positions, is particularly distributed throughout India, China, Japan, Siberia, etc. Young leaves, fruits, rhizomes and/or seeds thereof are edible. Especially, seeds (that is, Nelumbinis semen) are used to provide nutrition and tonic effects, sedative effects, etc., protect the spleen, provide anti-diarrheal effects, mental stability, etc., and/or treat chronic diarrhea, sexual dysfunction (sexual nervous breakdown) and leukorrhea, palpitation and insomnia, or the like. On the other hand, Nelumbo nucifera leaves are used to provide antipyretic effects and detoxification effects, while seeds and fruits of the foregoing plant are used to provide tonic effects and hemostatic effects, and/or treat enuresis and female diseases. Further, the roots are used to remove pathogenic heat, improve blood circulation or overall blood stasis, treat hematuria or gastrointestinal bleeding, and/or provide hemostatic effects, or the like.

Korean Patent No. 0063588 discloses a lotus (matured fruit of Nelumbo nucifera Gaertner) extract capable of protecting liver cells and preventing or treating liver damage, and a composition including the foregoing lotus extract as an active ingredient, which is useful for protecting liver cells, preventing and treating liver damage, and inhibiting mutation caused by liver cancer-inducing substances (that is, carcinogens).

In order to relieve hangovers, a number of domestic products have been developed and are commercially available. For example, Korean Patent No. 0181168 discloses a method for preparation of a natural tea for relieving hangover, using extracts of alder, sorbus and Ligustrum japonicum as well as Kudzu root extract; Korean Patent No. 10-0839586 discloses a method for preparation of a hangover relief drink using a water extract of Fugu xanthopterus, Hovenia dulcis Thunb fruits, red ginseng, black garlic, maca, curcuma longa Linne, Prunus mume fruits, Rubus fruticosus cv., Schizandra chinensis fruits and Kudzu root; and Korean Patent No. 0040515 discloses a method for preparation of a hangover relief beverage using seaweed extract, dried pollack extract, onion extract, an extract of bean sprouts, Oenanthe javanica extract and radish extract, with effects of activating alcohol dehydrogenase.

### SUMMARY OF THE INVENTION

Therefore, the present invention has been made in view of the above problems and the inventors have conducted studies to develop improved hangover relief composition and beverage, compared to conventional products. Accordingly, it is one object of the present invention to provide a composition for relieving and preventing hangover, prepared by adding an extract of Hovenia dulcis Thunb fruits as well as nicotinic acid amide to extracts of Rosa roxburghii, Nelumbo nucifera Gaertner and Engelgardtia chrysolepis HANCE, respectively, having favorable hangover relieving effects well known in the art by previous studies, wherein Hovenia dulcis Thunb fruits were known as the best one for liver detoxification according to traditional oriental medical texts such as Compendium of Materia Medica (Chinese: B\check{e}nc\check{a}o Gāngmù) and such liver detoxification effects after drinking alcohol have been objectively verified in various research documents, and wherein nicotinic acid amide is further added as a co-enzyme closely associated with in vivo alcohol (ethanol) metabolism. Another object of the present invention is to provide a beverage for relieving and preventing hangover, including the foregoing composition.

The foregoing objects have been accomplished by subjecting a composition for relieving and preventing hangover, which includes a Hovenia dulcis Thunb fruit extract or concentrate, extracts of Rosa Roxburghii, Engelgardtia chrysolepis HANCE and Nelumbo nucifera Gaertner, respectively, and nicotinic acid amide, to assessment of improvement in hangover extents through a hangover self-awareness efficacy evaluation procedure established by the present inventors.

In order to verify the efficacy of the composition for relieving and preventing hangover according to the present invention, self-awareness efficacy tests to determine whether actual consumers feel (or recognize) hangover relief or prevention effects before and after drinking, and objective efficacy results, wherein 83.7% of participants recognized improved effects of the composition in hangover relief or prevention, have been obtained. From corresponding tests, it can be confirmed that the novel beverage and composition for relieving and preventing hangover developed according to the present invention have excellent effects.

### BRIEF DESCRIPTION OF THE DRAWINGS

The above and other objects, features and other advantages of the present invention will be more clearly understood from the following detailed description taken in conjunction with the accompanying drawings, in which:

FIG. 1 illustrates assessment results of improvement in overall hangover extents after taking a composition for relieving and preventing hangover according to the present invention before drinking alcohol, based on results of self-awareness efficacy tests in relation to hangover relief using the composition for relieving and preventing hangover according to the present invention; and

FIG. 2 illustrates assessment results of improvement in different hangover items after taking a composition for relieving and preventing hangover according to the present invention before drinking alcohol, based on results of self-awareness efficacy tests using the composition for relieving and preventing hangover according to the present invention.

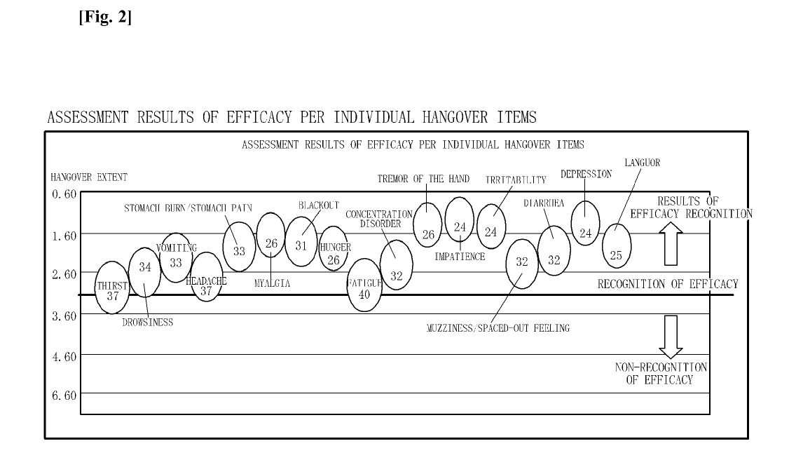

### DETAILED DESCRIPTION OF THE INVENTION

Now, the present invention will be described in more detail with reference to the following Examples. These examples are only provided to illustrate the present invention and should not be construed as limiting the scope and spirit of the present invention.

The present invention relates to a composition for relieving and preventing hangover and, more particularly, a composition for relieving and preventing hangover, which includes a Hovenia dulcis Thunb fruit extract or concentrate, nicotinic acid amide, a Rosa Roxburghii extract, an Engelgardtia chrysolepis HANCE extract and a Nelumbo nucifera Gaertner extract as active ingredients.

The present invention also relates to a beverage for relieving and preventing hangover, including the foregoing composition.

More specifically, a method for preparation of the composition for relieving and preventing hangover according to the present invention may include the following:

A Hovenia dulcis Thunb fruit extract concentrate may be prepared by adding 4 to 10 fold or more water to 100 g of Hovenia dulcis Thunb fruits and subjecting the mixture to extraction in hot water (95° C.) for 1 to 5 hours, resulting in an extract. After first filtration of the extract and sterilization thereof at 95° C. for 10 minutes or more, the same was subjected to re-filtration to obtain an extract. By concentrating the obtained extract to a solid content of 30 to 60%, the prepared concentrate may be used.

Instead of the Hovenia dulcis Thunb fruit extract concentrate prepared according to an exemplary embodiment of the present invention, an un-concentrated Hovenia dulcis Thunb fruit extract or another Hovenia dulcis Thunb fruit concentrate with different concentrations may also be employed. However, in such case, individual ingredients are added in predetermined amounts to satisfy the same solid content as that of Hovenia dulcis Thunb fruit extract concentrate (with a solid content of 55%) in an amount of 1.0 g, as described in the exemplary embodiment of the present invention.

More particularly describing an illustrative example, in order to replace 1 g of a Hovenia dulcis Thunb fruit extract concentrate (having a solid content of 55%) by a Hovenia dulcis Thunb fruit extract having a solid content of 4.5%, with reference to one serving dose proposed by the present invention, a proper amount of the latter may be estimated by the following equation.

That is, as a result of the following calculation:

solid content (55%) of a concentrate×content of the concentrate (1.0%)/solid

content (4.5%) of an extract,

an amount of the extract (having a solid content of 4.5%) required to become equal to 1.0% of the concentrate (having a solid content of 55%) may be about 12%.

In order to attain hangover relief (and prevention) efficacy as described above, a total amount of the composition comprising major active ingredients such as the Hovenia dulcis Thunb fruit extract concentrate (with a solid content of 55%) proposed according to the present invention, as well as extracts of Rosa Roxburghii, Engelgardtia chrysolepis HANCE and Nelumbo nucifera Gaertner, and nicotinic acid amide, would be better to become 1.353 g or more per day for an adult weighing 60 kg. The prepared composition may be taken without particular limitations. The number of taking the composition is not particularly limited. When the above Hovenia dulcis Thunb fruit extract concentrate (having a solid content of 55%) is replaced with the Hovenia dulcis Thunb fruit extract (having a solid content of 4.5%), the composition should contain the extract in the required amount of 12%, as described above, therefore, a total intake of the composition may become 12.353 g.

Each of respective extracts of Rosa Roxburghii, Engelgardtia chrysolepis HANCE and Nelumbo nucifera Gaertner is obtained by taking 200 g of a sample and adding 2 L of hot water (95° C.) thereto to conduct extraction for 2 hours. The extraction product is filtered to prepare about 400 g of an extract. After allowing the extract to settle for 12 hours, the supernatant is concentrated to form a concentrate having a solid content of 50%. The concentrate is spray dried, thereby obtaining extract powders of Rosa Roxburghii, Engelgardtia chrysolepis HANCE and Nelumbo nucifera Gaertner, respectively.

Nicotinic acid amide is biosynthesized from tryptophan, belongs to a vitamin type present in broad ranges of animal and plant species and is also known as vitamin B12. This is a co-enzyme of dehydrogenase participating in various kinds of oxidation/reduction reactions while being present in NAD+ and NADP+ forms in the human body. As shown below, the foregoing co-enzyme assists in activation of alcohol dehydrogenase and aldehyde dehydrogenase which are associated with decomposition of alcohol absorbed in vivo, thereby contributing to in vivo metabolism. Nicotinic acid amide is a component of a food raw material employed in a variety of applications and, in the case where the food raw material is a food grade raw material having a purity of at least 95%, may be applicable to any manufacturers irrespectively thereof.

Ethanol+NAD^{±}→acetaldehyde+NADH+H^{±}

(Alcohol Dehydrogenation)

Acetaldehyde+H₂O+NAD^{±}→acetic acid+NADH+H^{±}

(Acetaldehyde Dehydrogenation)

The composition for relieving and preventing hangover before and after drinking according to the present invention may comprise 0.27 to 5.3 parts by weight ('wt. part') of nicotinic acid amide, 4 to 20 wt. parts of Rosa Roxburghii extract, 0.4 to 5 wt. parts of Engelgardtia chrysolepis HANCE extract and 0.4 to 5 wt. parts of Nelumbo nucifera Gaertner extract, as active ingredients, in relation to 100 wt. parts of the Hovenia dulcis Thunb fruit extract described above.

With regard to the composition of the present invention, a Hovenia dulcis Thunb fruit extract concentrate may be replaced with a Hovenia dulcis Thunb fruit extract. However, in this case, as previously described, an amount of the foregoing extract must be determined such that a solid content of the extract becomes equal to that of the concentrate, in consideration of an amount of the concentrate to be used as well as a solid content of the same.

The inventive composition may be used as a pharmaceutical composition. In this regard, the composition may be made into a formulation optionally including a pharmaceutically acceptable carrier or additive, other than the foregoing active ingredients. Such a carrier or additive may include, for example, a diluent, a sweetener, a stabilizer, a flavoring agent, vitamins, an anti-oxidant, or the like.

The carrier or additive may be any pharmaceutically acceptable materials without being particularly limited thereto and, for example: a diluent such as lactose, a grain starch, etc.; a sweetener such as sucrose, liquid fructose, crystalline fructose, sucralose, sorbitol, aspartame, etc.; a stabilizer such as sodium carboxymethyl cellulose, beta-cyclodextrin, xanthan gum, etc., and so forth. In addition, in order to enhance taste of the composition, natural flavors such as fruit flavor, herbal flavor, etc., natural juice, a natural colorant such as flavonoids, a sweetening component such as fructose, honey, sugar, etc., an acidic agent (or a sour taste agent) such as citric acid, sodium citrate, etc., may be further added. Moreover, the inventive composition may further include supplementary ingredients, for example, vitamin B, C, E or beta-carotene, a mineral component such as Ca, Mg, Zn, etc., a phospholipid component such as lecithin, oligosaccharides, amino acid, taurine, or the like, other than the foregoing active ingredients.

Using the composition according to the present invention, in addition to beverages (or drinks), various formulations for relieving hangover such as liquid preparations (gels, extract), pills, powder, tablets, capsules, or the like may be manufactured for oral administration, without being particularly limited thereto.

Practical embodiments of the present invention will be described in detail according to the following examples.

### Example 1

### Preparation of Hovenia Dulcis Thunb Fruit Extract (Concentrate)

Hovenia dulcis Thunb fruit extract concentrate was prepared by adding 4 to 10 fold (that is, 400 to 1000 ml) or more of water to 100 g of Hovenia dulcis Thunb fruits and subjecting the mixture to extraction in hot water (95° C.) for 1 to 5 hours, resulting in an extract. After filtering the extract and sterilizing the same at 95° C. for 10 minutes or more, the sterilized material was subjected to re-filtration to obtain an extract. By concentrating the obtained extract to a solid content of 30 to 60%, the prepared concentrate may be used as a final extract concentrate of Hovenia dulcis Thunb fruits.

### Preparation of Rosa Roxburghii, Engelgardtia chrysolepis HANCE and Nelumbo nucifera Gaertner Extracts

After adding 2 L of hot water (95° C.) to 200 g of each of Rosa Roxburghii fruits, Engelgardtia chrysolepis HANCE leaves and Nelumbo nucifera seeds, respectively, extraction was conducted for 2 hours. Each extraction product was filtered to prepare about 400 g of an extract. After allowing the extract to settle for 12 hours without shaking, a supernatant thereof was concentrated to form a concentrate having a solid content of 50%. The concentrate was spray dried, resulting in extract powders of Rosa Roxburghii, Engelgardtia chrysolepis HANCE and Nelumbo nucifera Gaertner, respectively.

### Example 2

### Preparation of a Beverage Composition for Relieving Hangover Before and After Drinking

To a mixture of 10 g of the Hovenia dulcis Thunb fruit extract concentrate (having a solid content of 55%) (or 120 g of the Hovenia dulcis Thunb fruit extract having a solid content of 4.5%), 2 g of Rosa Roxburghii extract powder, 0.5 g of Engelgardtia chrysolepis HANCE extract powder and 0.5 g of Nelumbo nucifera extract powder, which were prepared in Example 1, 0.53 g of nicotinic acid amide, 100 g of glumate, 3.3 g of glutamic yeast extract, 20 g of taurine, 1 g of alanine, 25 g of refined sugar, 115.7 g of liquid fructose, 1.6 g of dL-maleic acid, 2.97 g of citric acid, 0.4 g of sodium citrate, 1.7 g of a flavoring agent (0.5 g of mixed fruit flavor 1, 0.8 g of mixed fruit flavor 2, 0.5 g of honey flavor) and 0.4 g of vita BD were added. Then, in order to make it edible, purified water was added to the mixture till a final volume thereof becomes 1 L. After stirring all ingredients until completely dissolved, high temperature short time sterilization (HTST, 98° C. for about 30 seconds) was conducted to eliminate microorganisms possibly remaining in the mixture. The sterilized product was introduced into a container and subjected to post-sterilization at a temperature of at least 80° C. for 5 to 10 minutes. Thereafter, the obtained product was cooled, labeled and packaged, thereby producing a hangover relief beverage (drink).

### Experimental Example 1

### Hangover Self-Awareness Efficacy Verification Test

A test procedure for hangover self-awareness efficacy was conceived and practically conducted on the basis of hangover assessment question items disclosed in a foreign document (Pittler, M. H. et al. CMAJ* 2003:169:1269-1273, Canadian medical association or its licensors), and an assessment procedure and data for hangover extents which are applied to “Research report regarding development of hangover relieving efficacy protocol” by the Korean food and drug administration (KFDA).

Age and gender distributions of panelists for the present test are shown in TABLE 1.

TABLE 1
Distribution of Test panelists

| Panel distribution | Male | Female | Subtotal |
|---|---|---|---|
| 29 years old or less | 2 | 6 | 8 |
| 30 to 34 years old | 7 | 8 | 15 |
| 35 to 39 years old | 9 | 1 | 10 |
| 40 to 44 years old | 9 | 0 | 9 |
| 45 years old or more | 1 | 0 | 1 |
| Total | 28 | 15 | 43 |

In respective test groups, 89% of men had an average drinking capacity of 3 bottles or less (of wine) while 86% of women had an average drinking capacity of 2 bottles or less (of wine).

After providing 100 ml (1 bottle) of the beverage composition for relieving and preventing hangover prepared in Example 2 according to the present invention to each of 43 panelists (28 men and 15 women), the panelists were allowed to sufficiently drink within individual drinking capacities. The following day after drinking, a questionnaire (that is, checklist) in regard to self-awareness for hangover relieving efficacy was given to each panelist to investigate hangover states and the panelist replied to questions on the checklist, to thereby conduct question assessment of self-awareness efficacy for hangover relief. Ingredients of the composition for relieving and preventing hangover developed according to the present invention, which was used in the foregoing assessment, are shown in TABLE 2 (see Example 2). In order to evaluate self-awareness efficacy for hangover relief, the checklist used in the present invention is shown in TABLE 3.

TABLE 2
Ingredients of the beverage composition product for relieving
and preventing hangover taken by test panelists

| Ingredients | Content % | Note |
|---|---|---|
| Hovenia dulcis Thunb fruit | 1.0 | When sing extract |
| extract concentrate (solid | | (solid content |
| content of 55%) | | of 4.5%), 12% |
| Rosa Roxburghii extract | 0.2 | |
| Engelgardtia chrysolepis | 0.05 | |
| HANCE extract | | |
| Nelumbo nucifera extract | 0.05 | |
| Yeast extract | 0.33 | |
| Nicotinic acid amide (Vit-B3) | 0.053 | |
| Taurine | 2.0 | |
| Glumate | 10 | |
| Alanine | 0.1 | |
| Refined sugar | 2.5 | |
| Liquid fructose | 11.57 | |
| Citric acid anhydride | 0.297 | |
| DL-maleic acid | 0.16 | |
| Vita BD | 0.04 | |
| Sodium citrate | 0.04 | |
| Flavor (mixed fruit flavor 1) | 0.05 | |
| Flavor (mixed fruit flavor 2) | 0.08 | |
| Flavor (honey flavor) | 0.05 | |
| Purified water | 71.43 | |
| Total | 100 | |

TABLE 3
Checklist for assessment of self-awareness efficacy for hangover relief

| | Hangover | | | | | |
|---|---|---|---|---|---|---|
| | relieving | Hangover extent | | | | |
| | efficacy | No - - - - - - - - - - - > Serious | | | | |
| Items | (Yes, No) | 1 | 2 | 3 | 4 | 5 |
| Thirst | | | | | | |
| Drowsiness | | | | | | |
| Vomiting/nausea/feeling sick | | | | | | |
| Headache | | | | | | |
| Stomach burn/abdominal pain/ | | | | | | |
| stomach pain | | | | | | |
| Myalgia | | | | | | |
| Short time blackout | | | | | | |
| Hunger | | | | | | |
| Fatigue/tiredness | | | | | | |
| Concentration decrease/ | | | | | | |
| concentration disorder | | | | | | |
| Tremor of hands and feet | | | | | | |
| Impatience/anger | | | | | | |
| Irritability | | | | | | |
| Muzziness/spaced-out feeling | | | | | | |
| Diarrhea | | | | | | |
| Depression | | | | | | |
| Languor | | | | | | |
| Overall hangover symptoms | | | | | | |

Assessment standards for scores given by panelists

(1) No hangover: no difficulty (or trouble) in daily life

(2) Little hangover: There is scarcely any difficulty (or trouble) in daily life

(3) Being usual: hangover is less than ordinary times and no difficulty (or trouble) in daily life

(4) General hangover: difficulty (or trouble) in daily life

(5) Heavy hangover: hangover is serious to have difficulty (or trouble) in daily life

As a result of self-awareness efficacy assessment, it was found that improvement effects (decrease in hangover symptoms) were recorded in most question items regarding such hangover symptoms when the panelists took the composition for relieving and preventing hangover according to the present invention. Specifically, with regard to important symptom items showing high response rate (75% or more) such as fatigue, headache, thirst, stomach burn/stomach pain, drowsiness, vomiting, etc., among hangover assessment items, the panelists reported good effects in a range of at least 70% and up to 85%. Therefore, it can be understood that the inventive composition for relieving and preventing hangover exhibits remarkably improved effects in relieving hangover. With regard to other items such as tremor of the hand, impatience, irritability, languor, Myalgia, etc., the inventive composition also exhibited improved effects. However, since the foregoing items have relatively decreased response rate by the panelists, compared to other items, it was considered that symptoms in these items are not major hangover symptoms. Detailed test results are shown in TABLE 4.

TABLE 4
Assessment results of self-awareness efficacy of different hangover symptoms

| | | | | | | Stomach burn/ | | | |
|---|---|---|---|---|---|---|---|---|---|
| Section | | Thirst | drowsiness | vomiting | headache | stomach pain | fatigue | Myalgia | Black out |
| Total | Level of | 2.84 | 2.50 | 2.12 | 2.57 | 1.85 | 2.80 | 1.54 | 1.71 |
| (43) | cognitive | | | | | | | | |
| | symptoms | | | | | | | | |
| | Response | 86% | 79% | 77% | 86% | 77% | 93% | 60% | 72% |
| | rate % | | | | | | | | |
| Men | Level of | 2.61 | 2.76 | 2.30 | 2.43 | 1.85 | 2.77 | 1.56 | 2.10 |
| (28) | cognitive | | | | | | | | |
| | symptoms | | | | | | | | |
| | Response | 53% | 49% | 47% | 53% | 47% | 60% | 42% | 47% |
| | rate % | | | | | | | | |
| Women | Level of | 3.21 | 2.38 | 2.08 | 3.07 | 1.92 | 3.00 | 1.56 | 1.45 |
| (15) | cognitive | | | | | | | | |
| | symptoms | | | | | | | | |
| | Response | 33% | 30% | 30% | 33% | 30% | 33% | 21% | 26% |
| | rate % | | | | | | | | |

| | | | | Tremor | | | | | |
|---|---|---|---|---|---|---|---|---|---|
| | | | Concentration | of the | | | | | |
| Section | | Hunger | disorder | hand | Impatience | Irritability | Muzziness | Diarrhea | Languor |
| Total | Level of | 1.88 | 2.31 | 1.28 | 1.17 | 1.33 | 2.31 | 1.94 | 1.81 |
| (43) | cognitive | | | | | | | | |
| | symptoms | | | | | | | | |
| | Response | 60% | 74% | 58% | 56% | 56% | 74% | 74% | 60% |
| | rate % | | | | | | | | |
| Men | Level of | 2.00 | 2.45 | 1.39 | 1.28 | 1.39 | 2.35 | 2.00 | 1.88 |
| (28) | cognitive | | | | | | | | |
| | symptoms | | | | | | | | |
| | Response | 44% | 47% | 42% | 42% | 42% | 47% | 47% | 40% |
| | rate % | | | | | | | | |
| Women | Level of | 2.14 | 2.17 | 1.25 | 1.00 | 1.29 | 2.42 | 2.25 | 1.56 |
| (15) | cognitive | | | | | | | | |
| | symptoms | | | | | | | | |
| | Response | 16% | 28% | 19% | 16% | 16% | 28% | 28% | 21% |
| | rate % | | | | | | | | |

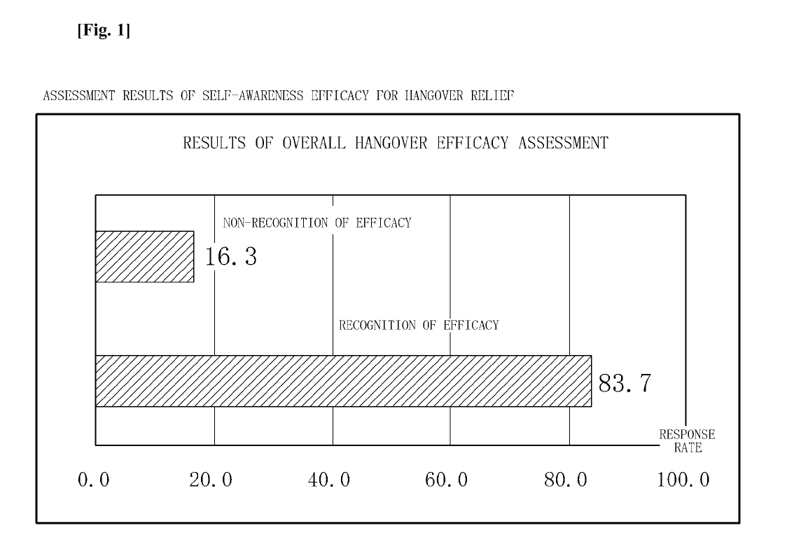

Moreover, as a result of confirming whether the composition for relieving and preventing hangover according to the present invention has effects of relieving or improving overall hangover symptoms, 83.7% (36 persons) among 43 panelists who participated in the present test recognized hangover relieving efficacy of the inventive composition, as shown in FIG. 1. Consequently, it can be identified that products developed according to the present invention may have excellent effects in relieving hangovers.

As apparent from the foregoing, the composition for relieving and preventing hangover which includes a Hovenia dulcis Thunb fruit extract or concentrate, extracts of Rosa Roxburghii, Engelgardtia chrysolepis HANCE and Nelumbo nucifera Gaertner, respectively, and nicotinic acid amide, as well as the hangover relief and prevention beverage including the same according to the present invention may exhibit excellent effects in hangover relief and prevention, as demonstrated from self-awareness efficacy tests.

Although the preferred embodiments of the present invention have been disclosed for illustrative purposes, those skilled in the art will appreciate that various modifications, additions and substitutions are possible, without departing from the scope and spirit of the invention as disclosed in the accompanying claims.

1. A composition for relieving and preventing hangover, comprising a Hovenia dulcis Thunb fruit extract concentrate, nicotinic acid amide, a Rosa Roxburghii extract, an Egelgardtia chrysolepis HANCE extract and a Nelumbo nucifera Gaertner extract as active ingredients.

2. The composition according to claim 1, wherein the composition includes 0.27 to 5.3 wt. parts of nicotinic acid amide, 4 to 20 wt. parts of the Rosa Roxburghii extract, 0.4 to 5 wt. parts of the Egelgardtia chrysolepis HANCE extract and 0.4 to 5 wt. parts of the Nelumbo nucifera Gaertner extract in relation to 100 wt. parts of the Hovenia dulcis Thunb fruit extract concentrate, as active ingredients.

3. The composition according to claim 1, wherein the Hovenia dulcis Thunb fruit extract concentrate is a Hovenia dulcis Thunb fruit concentrate having a solid content of 30 to 60 wt. %.

4. The composition according to claim 1, wherein the Hovenia dulcis Thunb fruit extract concentrate is a Hovenia dulcis Thunb fruit extract contained in an amount required to provide the same solid content as that of the Hovenia dulcis Thunb fruit extract concentrate.

5. The composition according to to claim 1, wherein the composition is in a liquid form.

6. A beverage for relieving and preventing hangover, comprising the liquid composition as set forth in claim 5 and a pharmaceutically acceptable carrier or additive further added thereto.
